(19) United States
(12) Patent Application Publication
(10) Pub. No.: US 2008/0312574 A1
(43) Pub. Date: Dec. 18, 2008

(54) Dressing Provided With a Thin Film Applicator

(75) Inventor: Jean-Marc Pernot, Dijon (FR)

Correspondence Address: MERCHANT & GOULD PC, P.O. BOX 2903, MINNEAPOLIS, MN, 55402-0903, US

(73) Assignee: Laboratoires Urgo, Chenove (FR)

(21) Appl. No.: 12/084,676

(22) PCT Filed: Nov. 16, 2006
(86) PCT No.: PCT/FR2006/051185

§ 371 Date: May 6, 2008

(30) Foreign Application Priority Data
Nov. 17, 2005 (FR) 0511646

(51) Int. Cl. A61F 13/02

(52) U.S. Cl. 602/52

(57) ABSTRACT

The present invention provides a novel system for applying dressings, said dressings comprising a thin film (1) coated on one face with a pressure-sensitive adhesive (2) and said application system being constituted by a detachable combination of another thin film (4) and a frame (5), thus avoiding problems with degradations to the adhesive bond between the skin and the dressing.

### FIELD OF THE INVENTION

The present invention relates to a novel system for applying dressings.

### PRIOR ART

Dressings with thin films, generally transparent, are widely used as a protective layer over wounds since they facilitate healing in a moist environment, while acting as a barrier against liquids and contamination by bacteria. Such films are also used as surgical drapes because of their ability to act as a barrier against bacterial contamination. Dressings and surgical drapes as described above are sold under the following trade names: TEGADERM® (3M, St Paul, Minn.) described in European patent EP-A-0 051 935 and OPSITE® (T.J. Smith & Nephew, Hull, England).

The polymer films used in such dressings are conformable, i.e. the films are sufficiently thin, flexible and pliable to be able to adapt well to the topology of the surface onto which they are positioned. Before use, the films have a detachable protective layer covering the surface of the film which is coated with adhesive. When the layer is stripped off, the adhesive-coated film, also termed the support, tends to crumple and stick to itself, thus preventing aseptic and gentle application of the dressing to the skin of a patient. Various systems for applying such products have been proposed to attempt to overcome this problem.

The principle of those various systems consists in adding to the coated film a additional layer of a rigid material either in the form of a uniform layer such as a film, or in the form of a frame, the rigid material facilitating positioning and being removed after the dressing has been applied to the skin.

However, while to a certain extent they are helpful for positioning, such solutions give rise to numerous problems when removing the layer of rigid material.

The difference in rigidity between the thin film and additional layer that is adhesively bonded, thermobonded, or mechanically fastened onto the surface of the film opposite from its surface coated in adhesive gives rise, during removal of the additional layer, to a disturbance in the adhesive connection between the skin and the dressing. That may cause partial detachment of the dressing from the skin or the appearance of wrinkles that can result in premature failure of the adhesion of the dressing or in poor application. Thus, with dressings in which a uniform rigid layer is bonded to the whole top face of the support, there is no means for absorbing the force exerted on removing that rigid layer in a movement in a single direction, which changes the quality of the adhesive bond between the dressing and the patient's skin.

Similarly, with dressings in which the rigid layer is a frame, when it is to be detached from the perimeter of the dressing, the force that is applied during the movement used to detach the peripheral frame in contact with the thin film also degrades the quality of the adhesion between the dressing and the skin.

Another disadvantage of such frame dressings is that they are not sufficiently rigid: after removal of the protective layer from the adhesive, they have a tendency to curl up, rendering positioning difficult.

While those two major types of dressings have been in existence for many years, no solution has yet been found for absorbing the force imposed during removal of the applicator portion via the rigid layer.

### Aim of the Invention

The aim of the invention is to provide a dressing that can absorb the forces exerted during removal of the applicator without degrading the rigidity properties of the dressing/applicator assembly during positioning on the skin of a patient.

In a first aspect of the invention, this aim is achieved by a composite adhesive dressing in accordance with the invention that comprises:
a support constituted by a thin pliable film with a top face and a bottom face;
a pressure-sensitive adhesive applied to at least a portion of the bottom face of the support;
a protective layer detachably applied to the pressure-sensitive adhesive opposite the support; the dressing being characterized in that it further comprises:
a bonding layer comprising a top face and a bottom face that is detachably assembled via its bottom face to the top face of the support opposite the adhesive; and
rigidification means comprising a top face and a bottom face, and being fixed at least in part to the periphery of the dressing on the top face of the bonding layer via a fastener means, leaving the central zone of said bonding layer not covered by said rigidification means, said rigidification means and said layer providing the support with rigidity, the bonding force between the support that is covered at least in part with adhesive and the skin being greater than the bonding force between the support and the bonding layer, and the bonding force between the rigidification means and the bonding layer also being greater than the bonding force between the bonding layer and the support.

It can be understood that this composite dressing of the present invention solves these problems by interposing between the rigidification means, for example a frame, and a thin film termed the support, a bonding layer that is in fact a second film such as a thin pliable film, for example, that will be eliminated at the same time as the rigidification means and therefore avoids problems associated with degradation of the adhesive bond between the skin and the dressing.

This additional layer can reduce and absorb the forces that are exerted on the dressing during removal of the rigid layer and can eliminate the above-mentioned problems associated with dressings using a rigid layer as an application means, and in particular a frame that covers the periphery of the dressing.

The dressing of the present invention can produce a delaminating force between the bonding layer and the thin film that is lower than with existing dressings (the dressing adheres to the skin without risk of removal during delamination of the thin film and the additional layer) while keeping the dressing more rigid following removal of the protective layer from the adhesive, thereby facilitating positioning.

Thus, after removing the protective layer covering the adhesive, the dressing is attached and then the assembly constituted by the rigidification means, e.g. a frame, together with the bonding layer is removed in order to achieve problem-free attachment of the final dressing constituted by the support and the adhesive. This can be achieved because the rigidification means, and in particular the frame, stiffens the dressing before it is positioned, and the thin film makes it possible using a simple movement in one direction only, to avoid problems of the bond between the skin and the adhesive degrading as otherwise arise when it is only a frame that is removed along the entire periphery of the dressing in a circular movement in which the action of the removal forces is different, or when it is a rigid additional layer that is removed after fixing to the skin of the patient.

Further, rigidification of the novel dressing results both from the bonding layer and the rigidification means that extend at least in part over the periphery of the dressing but leave the central zone of the bonding layer uncovered. Thus, differential rigidification of the dressing is obtained. The rigidification means, for example a frame, provide greater rigidity than the bonding layer, and the bonding layer in the central portion of the dressing provides limited but sufficient rigidification to allow the support to be smoothed over the skin of the patient, avoiding the formation of wrinkles. In contrast, the rigidification means prevent the edges of the dressing from “curling up”.

When the dressing acts to fasten a catheter or a like device on the skin of a patient, the rigidity in the central portion of the dressing is sufficiently reduced for the dressing, with its rigidification means, to conform to the shape of the catheter, and thereby provide effective fastening thereof, despite the presence of the rigidification means that are limited to the periphery of the dressing.

In a preferred embodiment of the present invention, the dressing has a tab associated with the frame that facilitates removal by inviting the person positioning the dressing to execute an optimal movement for withdrawing the additional layer, thereby further encouraging absorption of the forces exerted during removal of the application system.

Also preferably, the rigidification means cover at least a portion of the periphery of the bonding layer, producing a central aperture therein. Preferably again, these rigidification means form a frame.

In another preferred embodiment of the invention, the dressing comprises an absorbant layer that covers at least part of the adhesive.

In a second aspect of the invention, the dressing comprises a support at least partially covered on its bottom face with an adhesive and on its top face with an applicator. The dressing is characterized in that:
the bonding force between the support (1) and the applicator is 5 cN/cm [centiNewton/centimeter] to 25 cN/cm, preferably 8 cN/cm to 15 cN/cm;
the glass plate bonding force of the support (1) at least partially covered with adhesive (2) is 80 cN/cm to 200 cN/cm, preferably 100 cN/cm to 150 cN/cm; and
the rigidity angle α of the dressing, measured using the bending test, is 300 to 600, preferably 350 to 55°.

### DESCRIPTION OF THE FIGURES

Other characteristics and advantages of the invention become apparent from the following description of a number of embodiments of the invention. The description refers to the accompanying drawings in which:

FIG. 1 is a top view of a dressing in accordance with the present invention;

FIG. 2A is a cross section on line I-I of a first embodiment in accordance with the present invention in which the adhesive 6 covers the whole of a frame 5;

FIG. 2B is a cross section on line I-I of a second embodiment in accordance with the present invention in which the adhesive 6 is present only in portions A and B of a frame 5;

FIG. 3A is a cross section on line II-II of a third embodiment of the invention, in which the protective layer 3 is longer than the other layers forming the dressing;

FIG. 3B is a cross section on line II-II of a fourth embodiment in accordance with the present invention, in which the adhesive 6 is undercut;

FIG. 4 shows a variation in the dressing of the invention, showing the presence of a tab 11;

FIG. 5 shows a variation of the dressing of the invention, in which the dressing has an oval shape;

FIG. 6 shows a variation of the dressing of the invention, in which the frame covers only 3 peripheral edges;

FIG. 7 shows a variation of the dressing of the invention, showing the presence of a notch allowing the passage of tubes or a catheter;

FIGS. 8A, 8B, 8C and 8D are diagrams explaining how a dressing in accordance with the present invention is applied;

FIG. 9 is a diagram explaining the method of measuring rigidity (bending test).

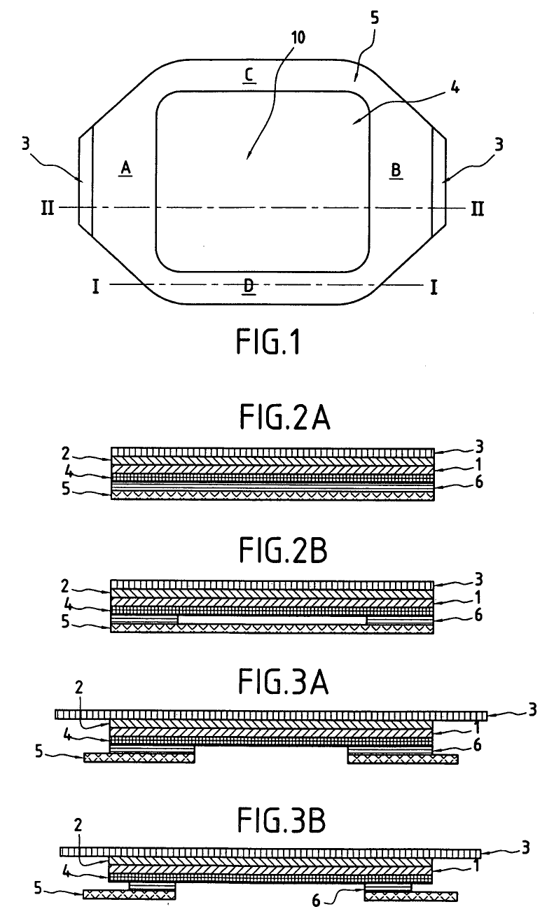

The structure of a composite adhesive dressing in accordance with the present invention is illustrated by reference to FIGS. 1, 2A and 2B that show respectively a top view and cross sections of the dressing on line I-I.

The composite adhesive dressing in accordance with the present invention is constituted by the following different elements:
a support 1 preferably formed by a thin, pliable and flexible film having a top face and a bottom face;
a pressure-sensitive adhesive 2 applied to at least a portion of the bottom face of the support 1;
a protective layer 3 detachably applied to the pressure-sensitive adhesive 2 opposite the support 1;
a bonding layer 4, comprising a top face and a bottom face, preferably constituted by a thin pliable film that is fixed via its bottom face to the top face of the support 1 opposite the adhesive 2, said support 1 and the bonding layer 4 being secured to each other using any fastening means;
rigidification means that, in the example described, are constituted by a frame 5 comprising a top face and a bottom face that is fastened at least in part to the periphery of the dressing on the top face of the bonding layer 4 via a fastener means 6, said means 6 being present at least in part on the periphery of the top face of the bonding layer 4 corresponding to the frame 5. The bonding layer 4 and the rigidification means 5 form the applicator.

More precisely, FIG. 1 is a top view of a dressing, showing the frame 5 that forms an aperture 10 in which there can be seen the top face of the bonding layer 4. The solid lines represent the right and left limits of the frame allowing the protector 3 to be seen, which in this embodiment is longer than the frame 5.

In a variation, the fastener means 6 of the dressing described above may be present only on the two widest sides A and B (shown in FIG. 1) between the frame 5 and the top face of the bonding layer 4.

The rigidification means never cover the central zone of the bonding layer.

FIG. 2A is a cross section on line I-I in which the support 1 is entirely coated with adhesive 2 that is covered with the protective layer 3. FIG. 2B shows the same section of a dressing in which the fastener means 6 is only present on the wide portions A and B of the frame 5.

As can be seen in FIG. 3A, which is a cross section on line II-II, the frame 5 may extend beyond the surface defined by the combination of layers 1, 2, and 4 to allow the dressing to be manipulated without touching the adhesive layer 2 following elimination of the protective layer 3.

In another optimized version of the dressing, as shown in FIG. 3B, a dressing may be produced in which the adhesive layer 6 is undercut to prevent creep of this adhesive in the event of pressure being applied to the dressing which could result in the dressing becoming stuck in its packaging.

The adhesive-free bottom face of the frame 5 that thus extends beyond the three preceding layers, may be detachably fastened to the top face of the protective layer 3 that also extends beyond the three layers 1, 2 and 4 and thus allows ready separation thereof before applying the dressing to the skin.

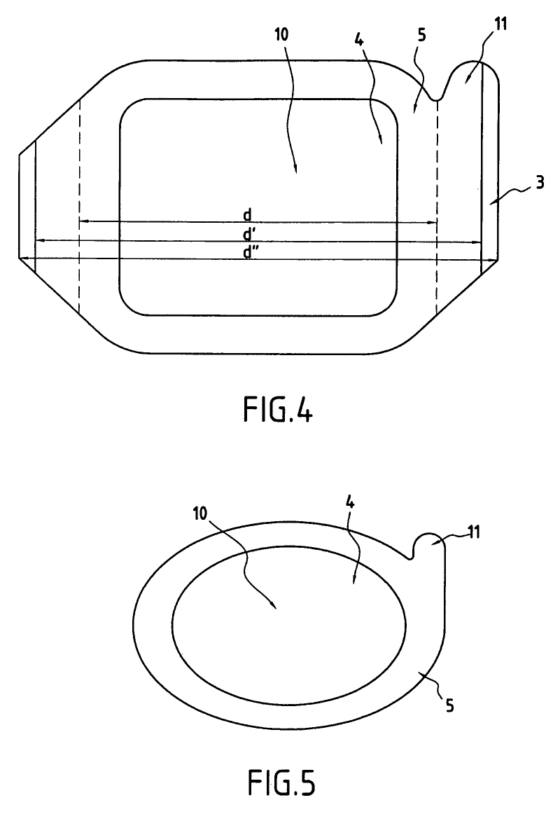

Using a dressing in accordance with the present invention is facilitated by the presence of a tab 11 (FIG. 4): after removing the protective layer covering the adhesive, the dressing is fastened to the skin, for example, then the assembly constituted by the frame 5 and the additional layer 4 is eliminated to achieve problem-free fastening of the final dressing constituted by the support 1 and the adhesive 2.

The composite adhesive dressing of the invention may have different shapes: square, rectangular (as shown in FIGS. 1 and 4), or oval (as shown in FIG. 5), to adapt it better to its various applications.

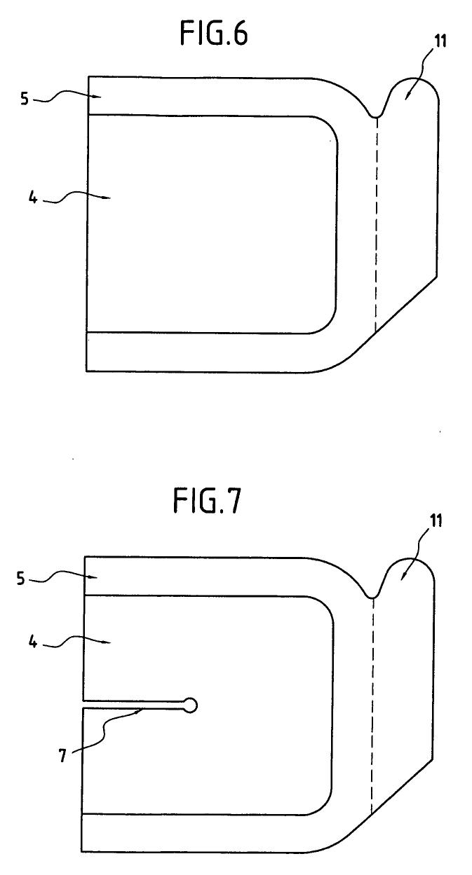

In another version of the composite adhesive dressing of the invention shown in FIGS. 6 and 7, the frame 5 does not cover the entire periphery of the dressing; it may, for example, be constituted only by three edges instead of four. The dressing may also exhibit a notch 7 to allow for the passage of tubes and catheters (FIG. 7).

The bottom face of the support 1 covered at least in part by the adhesive 2 may optionally include an absorbant layer. This absorbant layer is selected from the group composed of textiles based on: cotton; rayon; nonwoven fabrics; hydrocolloids; foams; or combinations of such elements. This absorbant layer may contain one or more substances selected from the group composed of: antimicrobial agents; drugs; chemical indicators; and combinations of such elements. If the dressing includes an absorbant layer, it is preferably positioned substantially in the center of the overall length of the dressing.

Production of the composite adhesive dressings of the invention calls upon elements that are routinely used in this field.

Thus, the configuration of the composite adhesive dressing of the present invention is useful in association with any conformable support comprising a pressure-sensitive adhesive coating applied to the support. Representative supports encompass: nonwoven textiles; woven textiles; knitted textiles; films; and other known materials that act as supports. This support is preferably conformable to anatomical surfaces.

The support 1 is preferably a pliable and flexible thin film. The term “thin” means a film with a thickness of 5 μm [micrometer] to 150 μm, preferably 15 μm to 70 μm. The term “pliable and flexible” means any material with sufficient conformability for it to adapt to curves on the body (such as joints) or a substrate (for example catheters). This film may optionally be a breathing film. It may optionally be watertight. Preferred films that may be used as the support 1 are: polyurethane; polyester or polyamide films; films based on a polyether-polyester copolymer (such as products sold by DuPont under the trade name Hytrel®); films based on polyester- or polyether-polyurethane copolymers (such as the products sold by Noveon with the trade name Estane®); or films based on polyether-polyamide copolymers (such as the products sold by Arkema with trade name Pebax®). Other polymers or copolymers may also be used to produce such films. The following may be mentioned in particular: polyethers; polyvinyl chlorides; polyvinylidene chlorides; polyvinyl alcohols; polyvinyl acetates; polystyrenes; polyolefins such as polyethylenes or polypropylenes, for example; polyvinyl fluorides; triblock or diblock styrene-olefin copolymers such as styrene/butadiene (such as the products sold with trade name Kraton®, for example); and block polyether amides. Combinations of said films may be used. Preferably, the support is transparent or translucent to facilitate positioning of the composite adhesive dressing.

The pressure-sensitive adhesives 2 that may be used in the present invention are any adhesives normally used for application to the skin, in particular hypoallergenic adhesive masses. Such adhesives are described in the “Handbook of pressure-sensitive adhesive technology”, third edition, Donates Sates, chapter 13-22. It is possible to use pressure-sensitive adhesives based on: acrylic; polyurethane; silicone; natural rubber; ethylene-vinyl acetate copolymer; or block copolymers of the poly(styrene-isoprene-styrene) type. In the context of the present invention, emulsion, solvent phase, or UV-curable pressure-sensitive acrylic adhesives are preferably used. Preferably, UV curable pressure-sensitive acrylic adhesives are used, for example the products sold by BASF under the trade name AcResin® A258UV.

These adhesives are applied to the support 1 in quantities of 15 g/m² [grams/square meter] to 100 g/m².

The pressure-sensitive adhesive 2 may optionally contain one or more substances selected from the group composed of: antimicrobial agents; drugs or any active ingredient; infection indicators; allergenic substances; hydrocolloids that can absorb exudates; and combinations of such elements.

The protective layer 3 may be constituted by any protective peelable material that is in routine use by the skilled person to protect the adhesive layer before use of the dressing. It may be in the form of a film, for example a film of a polyolefin such as polyethylene or polypropylene, a film of polyester, but also a metal foil or a silicone paper.

The bonding layer 4 may be selected from the group formed by a film, a foam, or a mesh. Preferably, the bonding layer is transparent or translucent to facilitate positioning of the composite adhesive dressing. This bonding layer 4 may be produced from a polyolefin such as polyethylene or polypropylene. The bonding layer 4 is preferably constituted by a film of polyethylene. The polyethylene film is 5 μm to 150 μm thick, preferably 20 μm to 70 μm thick.

To facilitate positioning of the dressing when, for example, the support 1 is intended to cover a catheter, this bonding layer 4 may optionally have reduced rigidity at the aperture 10. This reduction in rigidity may be achieved by slitting the bonding layer or by any other means known to the skilled person, allowing the rigidity of the interior of the aperture to be varied.

The bond between the bonding layer 4 and the support 1 may be physical, physico-chemical or chemical in nature. The support 1 may be thermobonded to the bonding layer 4. Preferably, the support 1 and the bonding layer 4 are secured to each other by a blown-bubble extrusion procedure. In this procedure, the material is extruded by an annular die to obtain a tube that is pinched between drawing rollers. Air pressure is admitted into the sealed sheath that has been formed, with the aim of drawing it to the required thickness. The “bubble” that is formed is cooled by a flow of air, and then wound. In the present invention, a co-extruded bilayer is preferably used which is composed of a film of polyurethane (which constitutes the support 1) together with a film of polyethylene (which constitutes the bonding layer 4) obtained by a blown-bubble extrusion procedure.

The frame 5 may be constituted by any material that is capable of supplying a certain amount of rigidity to the bonding layer 4 or to the assembly constituted by the support 1, the adhesive 2, and the bonding layer 4, thereby enabling better application by facilitating positioning of the composite dressing of the invention. Examples of such materials that can be mentioned are: paper; card; a foam; a mesh; a nonwoven fabric; etc.

The frame 5 may also be created by flock coating on the bonding layer 4. The frame 5 may cover only a portion of the periphery of the dressing; its thickness and its width may vary. The frame 5 is attached to the bonding layer 4 via fastener means 6. The fastener means 6 may cover the whole of the frame 5 or only part thereof. A dressing may be envisaged in which only the two widest portions of the frame 5 are attached to the bonding layer 4.

The fastener means 6 may thus be any fastener means that is currently known and that provides a bonding force between the frame 5 and the bonding layer 4 that is greater than that existing between the bonding layer 4 and the support 1. Preferably, an adhesive is used. Said adhesive may be selected from any of the adhesives that are known to the skilled person.

In the present invention, the frame 5 and the bonding layer 4 of the composite dressing endow the assembly constituted by the support layer 1 and the adhesive 2 with rigidity, thereby preventing the dressing from curling up or wrinkling even after removal of the protective layer 3. This feature has an advantage that is not insubstantial, especially when positioning the dressing, when the operator removes the protective layer; the dressing can be kept horizontal using just one hand before applying it to the skin or to the substrate for which it is intended (compress, tube, catheter, etc).

In the above description, the rigidification means are constituted by a frame 5 that covers at least three sides of the support. However, the scope of the invention encompasses the rigidification means having some other shape. To fulfill their function, which is to keep the dressing substantially flat, the rigidification means must have a major length and a major width that correspond to the major length and the major width of the dressing to hold the edges thereof properly.

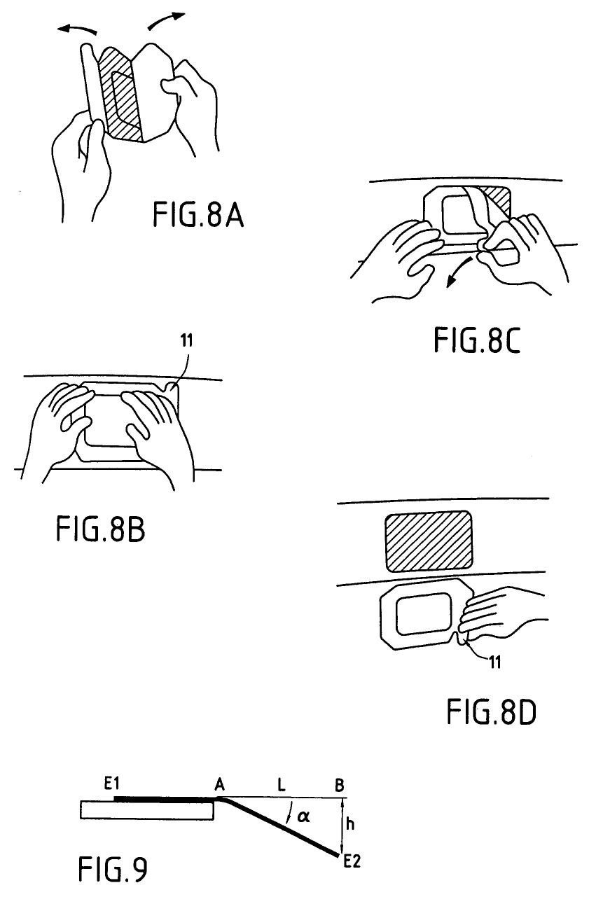

The easy way in which the dressing is used is illustrated in FIGS. 8A, 8B, 8C, 8D. The dressing is grasped by the two free edges of the protector 3 and of the frame 5, and then the protector 3 is removed (FIG. 8A). After removing the protective layer 3, the dressing may be held in just one hand, then it is applied to the site for which it is intended without touching the adhesive layer 2 with a hand (FIG. 8B). Next, the frame 5, to which the bonding layer 4 is attached, is removed (FIGS. 8C and 8D). This movement may be performed using the optional tab 11 that is present on the frame 5, as shown in FIG. 8D, thereby facilitating removal of the frame 5 and the bonding layer 4. The bond between the frame 5 and the bonding layer 4 provided by the fastener means 6 is such that no separation is possible during removal of these two layers after positioning the dressing. Similarly, the delaminating force (or bonding force) existing between the support 1 and the bonding layer 4 is much lower than the adhesive power existing between the support 1, the adhesive 2, and the skin or the substrate for which the dressing is intended. For this reason, the risk of detaching the dressing during removal of the bonding layer 4 and frame 5 is minimized.

Other characteristics and advantages of the invention become apparent from the following description of several embodiments of the invention.

Various samples of dressings of the invention were produced using conventional coating techniques and simple cutting operations, employing the following method.

The co-extrudate 1 constituted by a film of polyurethane attached to a polyethylene film using a blown-bubble extrusion procedure, was coated with a pressure-sensitive adhesive 2 using a conventional technique. Said adhesive 2 was covered with a protective layer 3. Next, the assembly was cut to the desired dimensions. The frame 5 was coated on one of its faces with an adhesive 6. The face of frame 5 that was coated with adhesive was covered with a protective film. The assembly constituted by the frame 5, the adhesive 6, and the protective film was cut to the desired dimensions. The protective film was then removed, and the assembly constituted by the frame 5 and the adhesive 6 was positioned on the polyethylene film 4.

The dressings so produced are described in the examples below.

During production of these dressings, the following were varied:
the weight (GSM) of the material constituting the frame 5;
the width of the frame 5;
the thickness of the bonding layer 4; and
the shape of the dressing.

The following materials were used:

The frame 5 was formed from paper with a GSM of 120 g/m² and a thickness of 100 μm, or 80 g/m² and a thickness of 110 μm. The support 1 was formed from 30 μm thick polyurethane with a GSM of 35 g/m², the bonding layer 4 was formed from polyethylene with a thickness of 30 μm and a GSM of 40 g/m² or 50 μm and a GSM of 46 g/m². The adhesive 2 used was a pure polyacrylate sold by BASF with the trade name AcResin® A258UV, 40±3 g/m² for all of the examples; the adhesive 6 between the frame 5 and the bonding layer 4 was a polyacrylate solution sold by Solutia with trade name Gelva® GMS 737, 40 g/m².

These dressings were rectangular in shape (as shown in FIG. 4). The dimension of the support 1 and the bonding layer 4 was 10×11.5 cm [centimeter] (length d); that of frame 5 was 10×14.5 cm (length d′); that of the protector 3 was 10×15.5 cm (length d″).

Various tests were carried out on the various dressings of the invention and on dressings sold with trade names TEGADERM® (3M, St Paul, Minn.) and OPSITE® (T.J. Smith & Nephew, Hull, England). TEGADERM® and OPSITE® are described in Examples 15 and 16.

The protocols for carrying out the tests were as follows:

### Rigidity Measurement Method

The bending test shown in FIG. 9 was used to measure the rigidity of the dressings of the invention. For this measurement, the dressings were freed of their adhesive protective layers and placed on the edge of a bench top 12 so that the length of the portion beyond the bench top was 11 cm long. The end E1 of the dressing was the end which adhered to the bench top; the end E2 of the dressing was that projected into space. Point A corresponded to the edge of the bench top to which the dressing adhered; point B corresponded to the vertical to the straight line E1A at E2. The length L, representing the distance between points A and B, was measured. The height h represented the distance between the end E2 of the dressing and the point B. The angle α was calculated as follows: tan α−1=h/L. In this method, the smaller the angle of α, the more rigid the material is considered to be. The measurements were carried out at 210±2° C. at a relative humidity of 60%±15%. Examples 1 to 13, 15 and 16 were tested.

### Method of Measuring Bonding Forces

Glass plate delaminating and adhesivity measurements were carried out on the dressing of the invention. To this end, the dressings were cut into 20 mm wide test pieces. These test pieces were placed on a glass plate. Next, two passes were made with a roller with a mass M=2 kg/cm [kilogram/centimeter] of dressing width. The test pieces were allowed to acclimatize at 21±2° C. and at a relative humidity of 61% for 10 minutes.

The delaminating force (or bonding force) of the bonding layer 4 to support 1 and the power of adhesion (or bonding force) of the support 1 and adhesive 2 to the glass plate were measured in succession using an electronic system that is capable of recording a force relative to a displacement (Synergie® 200, Adamel). The measurements were carried out with a 10 N probe at a rate of 100 mm/min. Examples 14, 15 and 16 were tested.

### EXAMPLE 1

The GSM of the paper used to prepare the frame 5 was 120 g/m²; the width thereof was 20 mm. The thickness of the bonding layer 4 formed from polyethylene was 50 μm. The dressing was rectangular in shape (as shown in FIG. 1).

### EXAMPLE 2

The GSM of the paper used to prepare the frame 5 was 120 g/m²; the width thereof was 20 mm. The thickness of the bonding layer 4 formed from polyethylene was 30 μm. The dressing was rectangular in shape (as shown in FIG. 1).

### EXAMPLE 3

The GSM of the paper used to prepare the frame 5 was 80 g/m²; the width thereof was 20 mm. The thickness of the bonding layer 4 formed from polyethylene was 50 Mm. The dressing was rectangular in shape (as shown in FIG. 1).

### EXAMPLE 4

The GSM of the paper used to prepare the frame 5 was 80 g/m²; the width thereof was 20 mm. The thickness of the bonding layer 4 formed from polyethylene was 30 μm. The dressing was rectangular in shape (as shown in FIG. 1).

### EXAMPLE 5

The GSM of the paper used to prepare the frame 5 was 120 g/m²; the width thereof was 12 mm. The thickness of the bonding layer 4 formed from polyethylene was 50 μm. The dressing was rectangular in shape (as shown in FIG. 1).

### EXAMPLE 6

The GSM of the paper used to prepare the frame 5 was 80 g/m²; the width thereof was 12 mm. The thickness of the bonding layer 4 formed from polyethylene was 30 μm. The dressing was rectangular in shape (as shown in FIG. 1).

### EXAMPLE 7

The GSM of the paper used to prepare the frame 5 was 80 g/m²; the width thereof was 12 mm. The thickness of the bonding layer 4 formed from polyethylene was 50 μm. The dressing was rectangular in shape (as shown in FIG. 1).

### EXAMPLE 8

The GSM of the paper used to prepare the frame 5 was 120 g/m²; the width thereof was 12 mm. The thickness of the bonding layer 4 formed from polyethylene was 30 μm. The dressing was rectangular in shape (as shown in FIG. 1).

### EXAMPLE 9

The GSM of the paper used to prepare the frame 5 was 120 g/m²; the width thereof was 8 mm. The thickness of the bonding layer 4 formed from polyethylene was 30 μm. The dressing was rectangular in shape (as shown in FIG. 1).

### EXAMPLE 10

The GSM of the paper used to prepare the frame 5 was 120 g/m2; the width thereof was 8 mm. The thickness of the bonding layer 4 formed from polyethylene was 50 μm. The dressing was rectangular in shape (as shown in FIG. 1).

### EXAMPLE 11

The GSM of the paper used to prepare the frame 5 was 80 g/m²; the width thereof was 8 mm. The thickness of the bonding layer 4 formed from polyethylene was 50 μm. The dressing was rectangular in shape (as shown in FIG. 1).

### EXAMPLE 12

The GSM of the paper used to prepare the frame 5 was 80 g/m²; the width thereof was 8 mm. The thickness of the bonding layer 4 formed from polyethylene was 30 μm. The dressing was rectangular in shape (as shown in FIG. 1).

### EXAMPLE 13

The GSM of the paper used to prepare the frame 5 was 80 g/m²; the width thereof was 12 mm. The thickness of the bonding layer 4 formed from polyethylene was 30 μm. The dressing was oval in shape (as shown in FIG. 5).

### EXAMPLE 14

The support 1 of the sample was formed from polyurethane with a thickness of 30 μm and the polyethylene bonding layer 4 was 30 μm thick; these two layers were attached using a blown-bubble extrusion procedure. The adhesive 2 was Acresin A258UV from BASF; the GSM of this mass was 40±3 g/m² and curing was carried out at 50 mJ/cm [milliJoule/centimeter]. The frame 5 was formed from 80 g/m² paper; the adhesive 6 was Gelva GMS 737 from Solutia, 40 g/m².

### EXAMPLE 15

The OPSITE® 10×12 dressing from Smith & Nephew was constituted by a polyurethane film with a thickness of 27 μm and a GSM of 28 g/m² and a polyethylene liner with a thickness of 70 μm and a GSM of 56 g/m². The GSM of the adhesive was 29 g/m².

### EXAMPLE 16

The TEGADERM® 10×12 dressing from 3M was constituted by a polyurethane film with a thickness of 22 μm and a GSM of 25 g/m² and a paper frame with a thickness of 120 μm and a GSM of 53 g/m². The GSM of the adhesive was 20 g/m².

The results of the rigidity measurements are shown in Table 1

TABLE I
Rigidity measurements

| | Tests | Angle α |
|---|---|---|
| Example 1 | 120 g/m² | 35° |
| | 20 mm | |
| | PE 50 μm | |
| Example 2 | 120 g/m² | 36° |
| | 20 mm | |
| | PE 30 μm | |
| Example 3 | 80 g/m² | 38° |
| | 20 mm | |
| | PE 50 μm | |
| Example 4 | 80 g/m² | 39° |
| | 20 mm | |
| | PE 30 μm | |
| Example 5 | 120 g/m² | 43° |
| | 12 mm | |
| | PE 50 μm | |
| Example 6 | 80 g/m² | 48° |
| | 12 mm | |
| | PE 50 μm | |
| Example 7 | 80 g/m² | 48° |
| | 12 mm | |
| | PE 50 μm | |
| Example 8 | 120 g/m² | 48° |
| | 12 mm | |
| | PE 30 μm | |
| Example 9 | 120 g/m² | 51° |
| | 8 mm | |
| | PE 30 μm | |
| Example 10 | 120 g/m² | 50° |
| | 8 mm | |
| | PE 50 μm | |
| Example 11 | 80 g/m² | 52° |
| | 8 mm | |
| | PE 50 μm | |
| Example 12 | 80 g/m² | 54° |
| | 8 mm | |
| | PE 30 μm | |
| Example 13 | 80 g/m² | 37° |
| | 12 mm | |
| | PE 30 μm | |
| Example 15 | OPSITE | >70° |
| Example 16 | TEGADERM | >70° |

The results obtained show that the rigidity of the OPSITE® and TEGADERM® product dressings was very low compared with the dressings of the invention. The differences between the values of the angles were of the order of 100 to 350 between the dressings of the invention and those two product dressings. The angle α could not be measured with precision because of the lack of rigidity of those two dressings. Further, the TEGADERM® dressing tended to curl up and retain that deformation. It can thus be seen that only the dressings of the invention have better rigidity after removing the protector.

The composite adhesive dressings of the present invention have an angle α e (obtained using this bending test) in the range 300 to 600, preferably 350 to 55°.

The results of the bonding force measurements are shown in Table II

TABLE II
Measurement of bonding forces

| | Example 14 | OPSITE | TEGADERM |
|---|---|---|---|
| Adhesive power (cN/cm) | 159 ± 4 | 156 ± 10 | 82 ± 2 |
| Delaminating force (cN/cm) | 9.6 ± 1.8 | 24 ± 1 | 28 ± 7 |
| Delaminating force to | 0.06 | 0.15 | 0.34 |
| adhesive power ratio | | | |

The dressing of the invention exhibited a low delaminating force compared with its adhesive power, thereby avoiding any disturbance to the adhesive bond between the dressing and the skin or any material onto which the dressing is applied during removal of the frame 5 and bonding layer 4. Thus, the ratio between the delaminating force and the adhesive power of the dressing of the invention is much lower than that obtained with the OPSITE® and TEGADERM® dressings. The composite adhesive dressings of the invention had a glass plate bonding force for the support 1 covered at least in part with adhesive 2 of 80 cN/cm to 200 cN/cm, preferably 100-cN/cm to 150 cN/cm, and a bonding force between the support 1 and the bonding layer 4 was 5 cN/cm to 25 cN/cm, preferably 8 cN/cm to 15 cN/cm.

Thus, the composite adhesive dressings of the invention have a low delaminating force while also having better rigidity. The problems with the dressing rolling up on itself and detachment of the support when removing the rigid layer of material after application of the dressing to the skin have been solved.

### Preferred Embodiment of a Dressing in Accordance with the Invention

The dressing of the invention is preferably constituted by a support 1 that is a polyurethane film with a thickness of 30 μm the bottom face of which is covered with an AcResin A258UV adhesive mass from BASF with a GSM of 40±3 g/m² and cured at 50 mJ/cm. The protective layer is formed from siliconized paper. The bonding layer 4 is formed from polyethylene with a thickness of 30 μm; the co-extrudate comprising the support 1 and the bonding layer 4 is obtained by a blown-bubble extrusion procedure. The frame 5 is formed from paper (120 g/m²); the width at segments C and D (shown in FIG. 1) is 120 mm. It is fixed to a support using adhesive 6 (Gelva GMS 737 from Solutia, 40 g/m²). As indicated in FIG. 4, the dressing has a tab 11 which facilitates removal of the bonding layer 4 and the frame 5 after positioning the dressing.

The dressings of the invention may be in the form of individual dressings with small dimensions or with larger dimensions depending on the use to which it is put. These dressings are thus be packaged individually into sealed pouches to guarantee they are stored in a sterile medium.

1. A composite adhesive dressing comprising:
a support comprising a thin pliable film with a top face and a bottom face;
a pressure-sensitive adhesive applied to at least a portion of the bottom face of the support;
a protective layer applied detachably to the pressure-sensitive adhesive opposite the support;
a bonding layer comprising a top face and a bottom face that is detachably assembled via its bottom face to the top face of the support opposite the adhesive; and
rigidification means comprising a top face and a bottom face, and being fixed at least in part to the periphery of the dressing on the top face of the bonding layer by fastener means, leaving the central zone of said bonding layer uncovered by said rigidification means, said rigidification means and said bonding layer providing the support with rigidity, the bonding force between the portion of the support covered at least in part with the adhesive and the skin being greater than the bonding force between the support and the bonding layer, and the bonding force between the rigidification means and the bonding layer also being greater than the bonding force between the bonding layer and the support.

2. A composite adhesive dressing according to claim 1, wherein said rigidification means cover at least a portion of the periphery of said dressing, forming an aperture in said central zone.

3. A composite adhesive dressing according to claim 2, wherein said rigidification means cover the whole of the periphery of the dressing, forming a frame.

4. A composite adhesive dressing according to claim 1, wherein glass plate bonding force of the portion of the support covered at least in part with adhesive is 100 cN/cm to 150 cN/cm, and the bonding force between the support and the bonding layer is 8 cN/cm to 15 cN/cm.

5. A composite adhesive dressing according to claim 1, wherein the rigidity angle α, measured by the bending test method, is 35° to 55°.

6. A composite adhesive dressing according to claim 1, wherein the support is selected from the group consisting of a film of polyurethane, polyester or polyamide.

7. A composite adhesive dressing according to claim 1, wherein the bonding layer comprises a film of polyethylene.

8. A composite adhesive dressing according to claim 1, wherein the rigidification means are fixed to the bonding layer by an adhesive or any other fastener means or formed directly on the bonding layer by flock coating.

9. A composite adhesive dressing according to any preceding claim claim 1 wherein the rigidification means are formed from paper, card, foam, mesh or any other material that is capable of endowing the bonding layer or the assembly comprising the support, the adhesive and the bonding layer with rigidity that is greater than that of the bonding layer.

10. A composite adhesive dressing according to claim 1, wherein the bottom of face of the support covered at least in part with the adhesive comprises an absorbant layer.

11. A composite adhesive dressing according to claim 1, wherein a co-extruded bilayer is used in the dressing, comprising polyurethane and polyethylene attached together using a blown-bubble extrusion procedure.

12. A composite adhesive dressing comprising a support having a bottom face that is covered at least in part with an adhesive and a top face covered with an applicator, characterized in that wherein:
the bonding force between the support and the applicator is 8 cN/cm to 15 cN/cm;
the glass plate bonding force of the support which is covered at least in part with adhesive is 100 cN/cm to 150 cN/cm; and
the rigidity angle α of the dressing, measured using the bending test, is 35 to 55.

13. A composite adhesive dressing according to claim 12, wherein the applicator comprises a bonding layer and rigidification means.

14. A composite adhesive dressing according to claim 13, wherein the rigidification means cover at least a portion of the periphery of the support.

15. Use of a composite adhesive dressing according to claim 1, comprising removing the protective layer, applying said dressing to the skin or any other substrate, then removing the assembly formed by the frame and the bonding layer.
